(19) United States
(12) Patent Application Publication
(10) Pub. No.: US 2023/0357726 A1
(43) Pub. Date: Nov. 9, 2023

(54) HUMAN PRIMARY MYELOFIBROSIS CELL STRAIN AND USE THEREOF

(71) Applicant: ZHEJIANG UNIVERSITY, Hangzhou (CN)

(72) Inventors: Jie JIN, Hangzhou (CN); Fenglin LI, Hangzhou (CN); Yile ZHOU, Hangzhou (CN); Wenjuan YU, Hangzhou (CN); Jinghan WANG, Hangzhou (CN); Xin HUANG, Hangzhou (CN); Yungui WANG, Hangzhou (CN)

(21) Appl. No.: 18/336,001

(22) Filed: Jun. 15, 2023

Related U.S. Application Data
(63) Continuation of application No. PCT/CN2022/080248, filed on Mar. 10, 2022.

(30) Foreign Application Priority Data
Jul. 22, 2021 (CN) 202110830647.7

(51) Int. Cl. C12N 5/09; G01N 33/50

(52) U.S. Cl. CPC C12N 5/0694; G01N 33/5011

(57) ABSTRACT

Providing a human primary myelofibrosis (PMF) cell strain, a construction method therefor and use thereof. The PMF cell strain is named as ZYXY-M2, and is deposited in China Center for Type Culture Collection (Wuhan University, Wuhan, China), with a preservation number of CCTCC NO: C202145. The present application is obtained by extracting and separating mononuclear cells from peripheral blood of a PMF patient and culturing in vitro for continuous natural passage. The leukemia cell strain is negative for JAK2, CALR, MPL mutation, positive for ASXL1, TP53, IKZF1, IDH1, FLT3 and TET1. The cell strain has good proliferation ability in vitro, and can be used as a cell material to study the pathogenesis of PMF and individualized treatment in vitro. Meanwhile, it can also be used to screen and evaluate drugs for in vitro and in vivo research of human PMF and guide clinical medication.

### CROSS-REFERENCE TO RELATED APPLICATIONS

The present application is a National Stage of International Application No. PCT/CN2022/080248, filed on Mar. 10, 2022, which claims priority to Chinese Patent Application No. 202110830647.7, filed on Jul. 22, 2021, both of which are hereby incorporated by reference in their entireties.

### TECHNICAL FIELD

The present application relates to the fields of biology and oncology, in particular to a human bone marrow fibrosis cell strain, a construction method therefor and use thereof.

### BACKGROUND

Malignant tumors whose blood system originates from bone marrow can be divided into acute myeloid leukemia and its related tumors and chronic myeloid tumors according to the differentiation level, proportion, cell morphology and molecular characteristics of primitive cells at the time of onset. The most common chronic myeloid tumors are chronic myeloid leukemia with positive BCR-ABL fusion gene and myeloproliferative tumor (MPN) with negative BCR-ABL. The most common diseases in MPN are polycythemia vera (PV), primary thrombocytosis (ET) and primary myelofibrosis (PMF). The results showed that the positive rates of JAK2, CALR and MPL mutations in PV, ET and PMF were 95%, 0% and 0% in PV patients, 60%, 20% and 3% in ET patients and 60%, 25% and 7% in PMF patients, respectively. Although there are many similarities among the three diseases at the gene level, the three diseases have obvious differences in bone marrow pathology, clinical manifestations, disease progression speed, clinical outcome and prognosis.

Primary myelofibrosis (PMF) is characterized by clonal bone marrow proliferation derived from stem cells in MPN, usually but not always accompanied by JAK2, CALR or MPL mutations, and about 10%-15% of them are negative for JAK2, CALR or MPL mutations. Clinical features include bone marrow reticulin/collagen fibrosis, abnormal inflammatory cytokine expression, anemia, hepatosplenomegaly, extramedullary hematopoiesis (EMH), systemic symptoms (such as fatigue, sweating at night, fever), cachexia, leukemia progression and shortened survival time. Compared with PV and ET, PMF has the worst prognosis. The research shows that the overall median survival time of the three diseases is: 20 years for PV, 14 years for ET and only 6 years for PMF. Up to now, five prognostic stratification systems have been proposed in the world for PMF, and the median survival time for high-risk PMF is less than 2 years. Causes of death include leukemia progression in about 20% of patients, and many patients also die from complications, including cardiovascular events and the consequences of cytopenia, including infection or bleeding.

At present, the treatment for PMF is extremely limited. Allogeneic hematopoietic stem cell transplantation (allo-HSCT) is the only method that can prolong the survival time of patients or cure them. However, at least 50% of patients who receive allo-HSCT have serious complications or death related to transplantation. Therefore, for individual patients, the risk of allo-HSCT must be weighed against the expected survival rate without allo-HSCT. Studies have shown that the 5-year disease-free survival rate (DFS) and treatment-related mortality rate (TRM) of PMF patients with matched related transplantation are 33% and 35% respectively, while unrelated transplantation is 27% and 50% respectively, and the recurrence rate is as high as 29%. Traditional drug treatment for PMF includes hydroxyurea and ruxolitinib, but it is limited to improving the clinical symptoms of patients, and has no anti-tumor activity, nor has it been proved that it can reverse bone marrow fibrosis or induce cytogenetics or molecular remission. Only 15%-25% of PMF patients responded to hydroxyurea and ruxolitinib, and the response time only lasted about one year. In recent years, new JAK inhibitors, such as fedratinib, momelotinib and pacritinib, are currently undergoing clinical trials, and they are only used as an alternative treatment for ruxolitinib resistance. It can be seen that the current drug treatment of PMF is only a palliative treatment. JAK2 mutation accounts for about 65% of PMF, and the therapeutic drugs for JAK2-negative patients need to be further explored.

The prognosis and prognosis of PMF are closely related to its molecular characteristics. The research shows that JAK2, CALR or MPL mutation may mediate the bone marrow fibrosis of PMF, but its transformation into leukemia may be related to other gene mutations, for example TET2, ASXL1, IDH1/2, EZH2, DNMT3A, CBL, RAS, IKZF1, TP53, SF3B1, SRSF2 and U2AF1 are all found in PMF. ASXL1, SRSF2, EZH2, IDH1, IDH2, U2AF1Q157 are included in the risk stratification system of PMF.

To sum up, the current PMF is the MPN with high leukemia rate, the worst prognosis and limited treatment. The basic tool of tumor research is tumor cell strain, because cell strain has unlimited proliferation ability in vitro and good tumorigenesis ability in vivo, which overcomes the limitation of primary cells. Tumor cells are also an effective tool for screening anti-tumor drugs and verifying tumor treatment plans. At present, no cell strain originated from PMF patients has been established at home and abroad. Cell strain K562 from CML and cell strain SET-2 from ET have been established abroad, which has greatly promoted the basic and clinical research of these two diseases. Therefore, it is urgent to establish a cell strain originated from PMF, which can be used to study the pathogenesis, molecular characteristics, new drug screening, and chemotherapy regimen update of PMF with the worst prognosis.

### SUMMARY

The objective of the present application is to provide a human primary myelofibrosis immortalized cell strain, a construction method therefor and use thereof aiming at the shortcomings of the prior art.

The objective of the present application is realized through the following technical solution: a human primary myelofibrosis cell strain, wherein the cell stain is named as a human myelofibrosis cell ZYXY-M2, which was deposited in China Center for Type Culture Collection on Jan. 20, 2021, with a preservation number of CCTCC NO: C202145.

The human primary myelofibrosis cells provided by the present application are negative for JAK2 mutation, negative for CALR mutation, negative for MPL mutation, positive for ASXL1 mutation, positive for TP53 mutation, positive for FLT3 mutation and positive for IKZF1 mutation. The cell morphology is primitive red blood cell, CD34 and CD11b are not expressed on a cell surface, but CD71 is expressed. The cells of the cell strain grow in suspension or with weak wall adherence.

The present application also provides the use of the human primary myelofibrosis cell strain as described above in any one or more of the following:
a. study on molecular characteristic and therapeutic mechanism of bone marrow fibrosis;
b. preparation of a tumor cell model or preparation of a tumor animal model; wherein, the tumor cell model includes progeny cells established by this cell strain or progeny cells established by this cell strain through transfection of fluorescent-labeled genes; the animal model includes animal models of PMF established by subcutaneous tumor bearing or tail vein injection;
c. screening and/or evaluation of tumor treatment drugs; wherein, the method of screening tumor therapy drugs can be as follows: adding different tumor therapy drugs into a culture medium of the human primary myelofibrosis cell strain, observing the changes of cell morphology, and obtaining preliminarily effective candidate drugs; then, applying the candidate drugs to the cells, and calculating the half inhibitory concentration (I50) of the screened effective drugs; selecting the drugs with the lowest IC50 to further act on animal models, and comparing the survival period, tumor size and metastasis of the animals with those in the non-application group, so as to screen potential drugs for treating PMF;
d. development of a tumor drug target;
e. preparation of tumor diagnosis products, such as flow antibodies with specific markers, gene detection kits, etc.
f. screening of a tumor biological therapy drug/reagent; wherein the tumor biotherapy drug/reagent is a tumor vaccine;
g. development of a bioengineering product related to tumor detection, wherein the tumor-related bioengineering product can be a PMF-specific molecular diagnostic PCR kit, a fluorescence in situ hybridization kit and the like.

The present application also provides a construction method for the human primary myelofibrosis cell strain, which is as follows:
obtaining fresh peripheral blood of a patient with primary myelofibrosis (PMF) diagnosed at the first time; adding 6 ml of the peripheral blood dropwise into a 15 ml sterile centrifuge tube with 6 ml lymphocyte separation solution added in advance; centrifuging at 2000 revolutions for 20 minutes; after centrifugation, taking the white cell precipitate of the mononuclear cell layer and adding to a new 15 ml sterile centrifuge tube, and adding 5 ml sterile 1×PBS resuspended cells, and centrifuging at 2000 revolutions for 5 minutes; after discarding the supernatant, adding a sterile red blood cell lysate to lyse the cells at room temperature for 5 minutes, then centrifuging at 2000 revolutions for 5 minutes; discarding the supernatant, adding 5 ml IMDM complete medium (IMDM 90%+fetal bovine serum 10%) to resuspend the cells, and centrifuging at 1500 revolutions for 5 minutes; discarding the supernatant, adding 5 ml IMDM complete medium to resuspend the cells; using a cell counting plate to count cells, adding 1*10⁸ cells into a 25 cm culture bottle, then adding an IMDM medium to 6 ml of mixed cells, and putting them into an incubator at 37 degrees Celsius with constant temperature and humidity to culture the cells; after 1 week, replacing the IMDM medium until the cells began to proliferate.

The present application has the beneficial effects that the human primary myelofibrosis cell strain of the present application can be passaged indefinitely, the cell shape in vitro is stable, and it accords with the biological characteristics of clinical tumors. The human primary myelofibrosis cell strain is originated from PMF patients, with JAK2 mutation negative, CALR mutation negative, MPL mutation negative, ASXL1 mutation positive, TP53 mutation positive, FLT3 mutation positive and IKZF1 mutation positive. The human primary myelofibrosis cell strain can be used to study the mechanism of the occurrence and development of PMF. The cells can also be used to analyze the curative effect of new anti-PMF drugs and combined schemes, to screen and evaluate PMF drugs, and to guide clinical medication. It is of great significance to reveal PMF, an MPN with poor prognosis.

### BRIEF DESCRIPTION OF DRAWINGS

The present application will be further explained with reference to the following examples and drawings.

FIG. 1 shows the results of staining of the human primary myelofibrosis cell strain Wright-Giemsa;

FIG. 2 is the expression chart of cell surface antigen of the human primary myelofibrosis cell strain;

FIG. 3 is a graph of cell growth of the human primary myelofibrosis cell strain at different cell densities;

FIG. 4 is the Circos diagram of genome variation of the human primary myelofibrosis cell strain.

### DESCRIPTION OF EMBODIMENTS

The present application will be illustrated with reference to the following examples, but are not limited thereto. The experimental methods without specific conditions in the following examples are usually in accordance with conventional conditions.

### Example 1 Preparation of a ZYXY-M2 Cell Strain

Primary cell culture: leukemia mononuclear cells were immediately isolated from the fresh high white blood cell isolated specimen (male, 62 years old, PMF, white blood cell 178.1*10⁹/L) obtained from the First Affiliated Hospital of Zhejiang University Medical College. In the bio-safety cabinet, 6 ml of a separation liquid was added dropwise into a 15 ml sterile centrifuge tube with 6 ml of lymphocyte separation liquid added in advance, and the mixture was centrifuged at 2000 revolutions for 20 minutes. After centrifugation, the mononuclear cell layer was added into a new 15 ml sterile centrifuge tube, and 5 ml of sterile 1×PBS resuspended cells was added, and the mixture was centrifuged at 2000 revolutions for 5 minutes. After discarding the supernatant, a sterile red blood cell lysate was added to lyse the cells at room temperature for 5 minutes, then the mixture was centrifuged at 2000 revolutions for 5 minutes. After discarding the supernatant, 5 ml of an IMDM complete medium (IMDM 90%+fetal bovine serum 10%) was added to resuspend the cells, and the mixture was centrifuged at 1500 revolutions for 5 minutes. After discarding the supernatant, 5 ml of the IMDM complete medium was added to resuspend the cells. A cell counting plate was used to count cells, and 1*10⁸ cells were added into a 25 cm culture bottle, and then the IMDM medium was added to 6 ml of mixed cells, and the mixture was put into an incubator at 37 degrees Celsius with constant temperature and humidity to culture the cells. After 1 week, the medium was replaced by a new IMDM medium to remove the cell debris, and the culture continued. The culture medium was changed once a week.

Cell subculture: Cell apoptosis occurred after 2 weeks of culture. The remaining non-apoptotic cells proliferated very slowly, and the culture medium was changed every week. When the cells were cultured for 2.5 months, they began to proliferate and grew in suspension. At this time, the cell culture medium was changed every 72 hours and the passage began. Up to now, the passage of cells has exceeded 50 generations, showing an immortalized cell strain.

According to the present application, the cells grow in a suspended state or are weakly adherent to the wall (without trypsin digestion), and grow in groups, and the cells are round or oval, and the growth rate of the cells is stable. The cells are named ZYXY-M2, and the cells are preserved in China Typical Culture Collection Center (address: Wuhan, Wuhan University, China, zip code 430072) on Jan. 20, 2021, with a preservation number of CCTCC NO: C202145.

### Example 2: Biological Characteristics and Application of a Human Acute Myeloid Leukemia Cell Line

According to the present application, an IMDM culture medium containing 10% fetal bovine serum was adopted to culture the cell strain, so that the cell strain could grow stably in vitro and be passaged stably. Microscopically, the cells were suspended or weakly adherent to the wall, growing individually or in groups, round or oval. According to Wright-Giemsa staining, the cells were mainly primitive red blood cells, with a large size, dark blue cytoplasm, a large number of vacuoles in some parts, a lightly stained area around the nucleus, fine granular nuclear chromatin and obvious nucleoli. The cell strain showed no expression of CD34 and CD11b, but high expression of CD71, as tested by flow cytometry. The whole exon sequencing of the cell strain showed that the cell strain negative mutations of JAK2, CALR and MPL, poor prognosis of PMF or positive mutations of TP53, ASXL1 and TKZF1 of genes related to leukemia transformation. In addition, it showed positive mutations of FLT3 and TET1 genes related to acute myeloid leukemia. The cell strain can be used for preparing tumor cell models or preparing tumor animal models; screening and/or evaluating/preparing tumor therapeutic drugs; developing tumor drug targets; preparing tumor diagnosis products; screening tumor biotherapy drugs/reagents; developing tumor-related bioengineering products, which are specifically as follows:

Morphological Observation

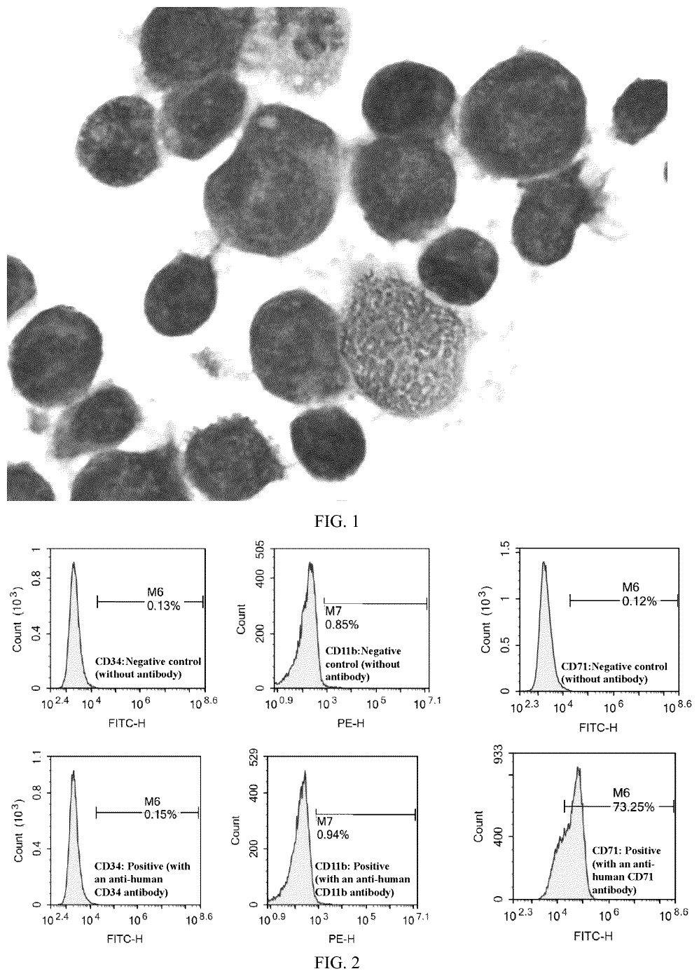

The cultured ZYXY-M2 cell strain was placed under an inverted microscope to observe that the cells grew in a clustered suspension or weakly adhered to the wall, and the cells were round or oval. 1*10⁶ cultured cells were added in a 1.5 ml EP tube, and were centrifuged at 1500 revolutions for 5 minutes. The supernatant was discarded, and 10 ul of a culture medium was added to suspend the cells, followed by smearing on a slide; after the cell smear was dried, the cell smear was stained with a Gareth-Giemsa staining solution for 5 minutes, then rinsed and dried. The cell morphology was observed under the inverted microscope. As shown in FIG. 1, the cells were mainly primitive red blood cells, with a large size, dark blue cytoplasm, a large number of vacuoles in some parts, a lightly stained area around the nucleus, fine granular nuclear chromatin and obvious nucleoli.

Cell Surface Antigen Detection

1*10⁶ of the cultured cells were taken and divided into three clean and sterile EP tubes, and centrifuged at 1500 revolutions. After 5 min, the supernatant was discarded and the cells were washed with 1×PBS. After centrifugation at 1500 revolutions, the supernatant was discarded after 5 min, and then 100 ul of 1×PBS resuspended cells was added into each EP tube, the first tube without antibody, the second tube with a FITC-labeled anti-human CD34 antibody and a PE-labeled anti-human CD11b antibody, the third tube with a FITC-labeled anti-human CD71 antibody, with each antibody being 10 ul; the mixture was incubated at room temperature for 30 min, 1 ml of 1×PBS was added, and the mixture was centrifuged at 1500 revolutions for 5 min. The supernatant was discarded, and 300 ul of 1×PBS was added into each tube to resuspend the cells, and then the expression of CD34, CD11b and CD71 was measured by an up-flow analyzer. As shown in FIG. 2, this cell strain does not express CD34 and CD11b antigens, but highly expresses the erythroid marker CD71(73.25%), which is consistent with the morphology of proerythroblasts.

Observation of Proliferation Ability In Vitro

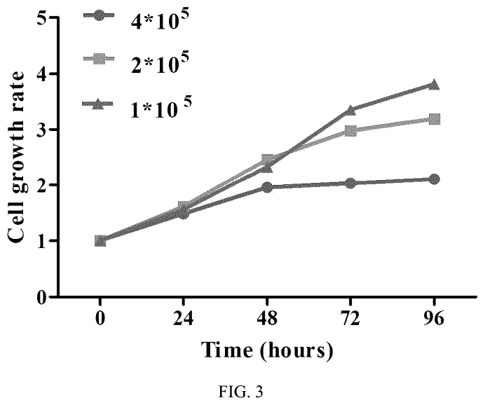

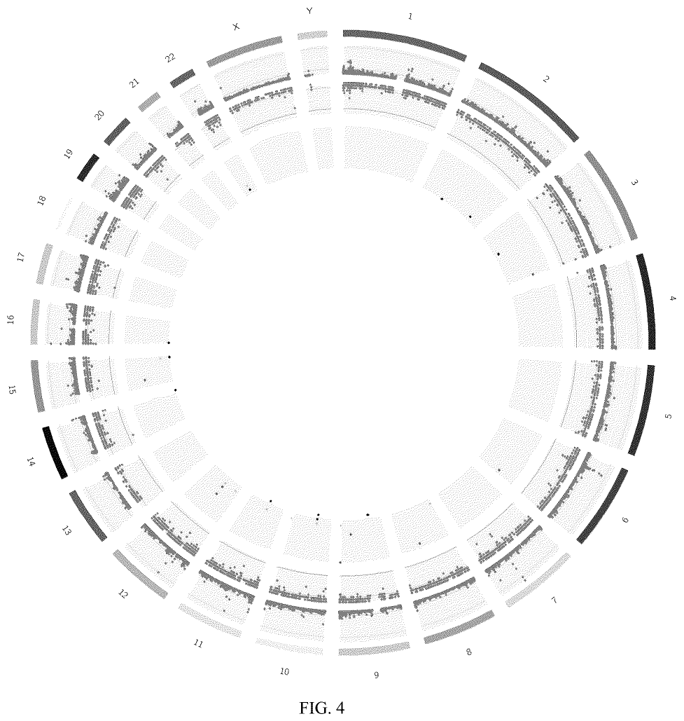

The cultured ZYXY-M2 cell strain was plated on the 96-well plate at the concentrations of 1, 2, 4*10⁵/ml, with 100 ul per well. At 1, 24, 48, 72 and 96 hours, 20 ul MTS was added, respectively. After 4 hours, the absorbance of the 96-well plate was measured by an enzyme-labeled instrument, and the proliferation curves of the cells at different plating concentrations were drawn as shown in FIG. 3. The cells of the cell line have good proliferation ability in vitro, showing malignant growth.

Whole Exon Sequencing of Cells

After counting the cultured cells, a cell culture fluid containing 5*10⁶ cells were centrifuged at 1500 revolutions for 5 min, the supernatant was removed leaving the cell mass; 1×PBS was added to resuspend the cells, followed by centrifugation at 1500 revolutions for 5 min, and the supernatant was removed, leaving the cell mass. The genomic DNA was extracted from the samples, and the DNA samples qualified for electrophoresis were randomly broken into fragments of 150 bp-220 bp by Covaris. The library was built and captured by an Agilent Sureselect Human All Exon V6 kit. The DNA fragments were repaired at the end, added with a ployA tail, followed by steps such as sequencing adapter, purification, capturing by magnetic beads and PCR amplification, and finally the library was built. After the library was tested to be qualified, double-ended sequencing was carried out by a sequencer. After the sequencing machine got the raw sequencing data (Raw data), it entered the bioinformatics analysis process, which was divided into two stages: 1, quality evaluation of sequencing data: whether the sequencing of the database had reached the standard was evaluated mainly by the statistics of sequencing error rate, data volume, comparison rate, coverage and the like, and follow-up analysis was carried out if the standard was met.

2, Variation information analysis: high-quality sequencing sequences were compared to the reference genome, and the variation information in the sample was detected, and the detected variation information was analyzed and interpreted. The genomic variation Circos is shown in FIG. 4. results of SNP and InDel mutation locus detection and analysis in ZYXY-M2 cell genome and mutation information closely related to hematological malignancies are summarized in Table 1. It can be known from the table that the cells of the cell strain showed negative mutations of JAK2, CALR and MPL, poor prognosis of PMF or positive mutations of TP53, ASXL1 and TKZF1 of genes related to leukemia transformation. In addition, it showed positive mutations of FLT3 and TET1 genes related to acute myeloid leukemia. It is suggested that the cells have certain value in the study of PMF transformation, including the study of the therapeutic mechanism or pathogenesis of PMF transformation into leukemia and the screening of targeted drugs for the above mutations.

TABLE 1
Summary results of SNP and InDel
mutation sites in ZYXY-M2 cells

| | Mutation | | Mutation |
|---|---|---|---|
| Gene name | site | Domain | type |
| JAK2 | — | | |
| CALR | — | | |
| MPL | — | | |
| LNK | — | | |
| TET2 | — | | |
| ASXL1 | C20: 31022959 T < C | Exon-non-synonymous | M |
| IDH1 | C2: 209120640 C < T | Upstream | M |
| IDH2 | — | | |
| EZH2 | — | | |
| DNMT3A | — | | |
| CBL | — | | |
| RAS | — | | |
| KRAS | — | | |
| HRAS | — | | |
| NRAS | — | | |
| IKZF1 | C7: 50430033 A < G | Exon-non-synonymous | M |
| TP53 | C17: 7579472 G < C | Exon-non-synonymous | WT/M |
| SF3B1 | — | | |
| SRSF2 | — | | |
| U2AF1 | — | | |
| FLT3 | C13: 28611358 C < T | Exon-non-synonymous | WT/M |
| | C13: 28624294 G < A | Exon-non-synonymous | WT/M |
| | C13: 28674628 T < C | Exon-non-synonymous | WT/M |
| TET1 | C10: 70332580 A < G | Exon-non-synonymous | WT/M |
| | C10: 70332672 T < G | Exon-non-synonymous | WT/M |
| | C10: 70332862 C < T | Exon-non-synonymous | WT/M |
| | C10: 70445539 A < G | Exon-non-synonymous | WT/M |
| | C10: 70405855 A < G | Exon-non-synonymous | WT/M |

Note:
M means that only mutant type is detected in the cell; WT/M means that both wild type and mutant type of the cell are detected.

Cell STR Identification

The cultured cells were sent to Shanghai Blowing for genotyping of the STR locus and Amelogenin site. The results suggested that the cell strain was unique because it did not match the existing cell strains in the world. Furthermore, the genotyping matching degree with STR locus and Amelogenin locus of the cell just isolated from the patient was up to 97%, and they were cells from the same source, that is, the culture process of the cell source was correct and there was no cross-contamination. The genotyping of STR and Amelogenin loci in cells is shown in Table 2.

TABLE 2
Genotyping results of STR locus and Amelogenin locus of cells

| | STR information of test cells | | |
|---|---|---|---|
| | Test cell name: ZYXY-M2 | | |
| Loci | Allele1 | Allele2 | Allele3 |
| D5S818 | 10 | 14 | |
| D13S317 | 8 | 9 | |
| D7S820 | 10 | 12 | |
| D16S539 | 11 | 13 | |
| VWA | 16 | 18 | |
| TH01 | 7 | 9.3 | |
| AMEL | X | Y | |
| TPOX | 8 | 11 | |
| CSF1PO | 12 | 13 | |
| D12S391 | 17 | 19 | |
| FGA | 21 | 22 | |
| D2S1338 | 23 | 25 | |
| D21S11 | 29 | 29 | |
| D18S51 | 14 | 16 | |
| D8S1179 | 14 | 15 | |
| D3S1358 | 16 | 17 | |
| D6S1043 | 11 | 14 | |
| PENTAE | 14 | 15 | |
| D19S433 | 14 | 14 | |
| PENTAD | 10 | 11 | |
| D1S1656 | 13 | 17 | |

The above examples are used to explain, rather than to limit the present application. Any modifications and changes made to the present application within the scope of protection of the spirit and claims of the present application fall within the scope of protection of the present application.

What is claimed is:

1. A human primary myelofibrosis cell strain, wherein the cell stain is named as a human myelofibrosis cell ZYXY-M2, which was deposited in China Center for Type Culture Collection on Jan. 20, 2021, with a preservation number of CCTCC NO: C202145.

2. The human primary myelofibrosis cell strain according to claim 1, wherein a cell morphology is primitive red blood cell, CD34 and CD11b are not expressed on a cell surface, but CD71 is expressed, and cells of the cell strain grow in suspension or with weak wall adherence.

3. The human primary myelofibrosis cell strain according to claim 1, wherein the cells are negative for JAK2 mutation, negative for CALR mutation, negative for MPL mutation, positive for ASXL1 mutation, positive for TP53 mutation, positive for IKZF1 mutation, positive for IDH1 mutation, positive for TET1 mutation and positive for FLT3 mutation.

4. A progeny cell of the human primary myelofibrosis cell strain according to any one of claim 1.

5. Use of the human primary myelofibrosis cell strain according to any one of claim 1 in any one or more selected from the following:
a. study on molecular characteristic and therapeutic mechanism of bone marrow fibrosis;
b. preparation of a tumor cell model or preparation of a tumor animal model;
c. in vitro screening and/or in vitro evaluation of a prepared tumor therapeutic drug;
d. development of a tumor drug target;
e. preparation of a tumor diagnosis product;
f. in vitro screening of a tumor biological therapy drug/reagent; and
g. in vitro development and detection of a tumor-related bioengineering product.

6. The use according to claim 5, wherein an animal for preparing the tumor animal model is an immunodeficiency mouse or a C57BL6 mouse.
